(19) United States
(12) Patent Application Publication
(10) Pub. No.: US 2026/0256372 A1
(43) Pub. Date: Sep. 3, 2026

(54) A SENSOR ARRANGEMENT FOR DETECTING AN INTRACRANIAL PRESSURE, AND A METHOD FOR DETECTING AN INTRACRANIAL PRESSURE

(71) Applicant: BRAINLINK AB, Askim (SE)

(72) Inventor: Dan FARAHMAND, Askim (SE)

(21) Appl. No.: 19/162,823

(22) PCT Filed: Mar. 8, 2024
(86) PCT No.: PCT/SE2024/050215

§ 371 Date: Sep. 5, 2025

(30) Foreign Application Priority Data
Mar. 8, 2023 (SE) 2350258-6

(51) Int. Cl. A61B 5/03; A61B 5/00

(52) U.S. Cl. CPC A61B 5/031; A61B 5/6864; A61B 2560/0214; A61B 2560/0223; A61B 2560/0257; A61B 2562/0219; A61B 2562/0247

(57) ABSTRACT

The present disclosure relates to a sensor arrangement (2) for detecting an intracranial pressure, said arrangement (2) comprising: a housing (8) defining an internal space (15a; 15b) which is configured for accommodating a pressure sensor device (9) and an electronic control unit (14) to which said pressure sensor device (9) is connected; and fastening means (7; 2b) cooperating with said housing (8) for mounting, optionally in a permanent manner, said arrangement (2) in a human skull bone (3). Furthermore, the housing (8) is arranged with an opening (11) which is covered by a membrane (12) being made of titanium or a similar thin and flexible material, and is configured for generating vibrations in accordance with an existing outer pressure, said vibrations being transferred to said pressure sensor device (9). The invention also relates to a method for detecting an intracranial pressure.

### FIELD OF THE INVENTION

The present invention relates to a sensor arrangement for detecting an intracranial pressure. The arrangement comprises a housing defining an internal space which is configured for accommodating a pressure sensor device and an electronic control unit to which said pressure sensor device is connected. Also, the arrangement comprises fastening means cooperating with said housing for mounting, optionally in a permanent manner, said arrangement in a human skull bone.

The disclosure also relates to a method for detecting an intracranial pressure, said method for detecting an intracranial pressure by means of a sensor arrangement. The method comprises: providing an internal space by means of a housing forming part of said sensor arrangement; accommodating, within said internal space, a pressure sensor device and an electronic control unit to which said pressure sensor device is connected; and mounting, optionally permanently, said arrangement in a human skull bone by means of fastening means cooperating with said housing.

### BACKGROUND OF THE INVENTION

In the field of medical science, there is a need for devices and methods for measuring the intracranial pressure in the brain of a person and for detecting pulsations of the brain. Such a need may arise as a consequence of certain types of neurogical disorders and injury to the brain, such as for example hydrocephalus. This condition corresponds to an abnormally high buildup of fluid in cavitites within the brain and can normally be treated by surgically inserting a drainage system for controlling the flow of fluid in the brain.

Furthermore, it is previously known to provide a pressure monitoring system in order to detect and monitor the intracranial pressure. Such a system can be implanted in the head of a patient and can be used together with the above-mentioned drainage system during treatment of hydrocephalus.

Based on the above, there is consequently a need for systems and methods for reliable and accurate measuring and monitoring of the intracranial pressure during certain medical conditions, such as for example hydrocephalus.

With reference to prior art, is should be noted that the patent document US 11422051 teaches an arrangement for sensing a pressure in a shunt for use during treatment of hydrocephalus. The arrangement is based on a pressure sensor which is arranged in a cavity within a housing. The housing is also configured with an inlet and an outlet which are used as parts of a hydrocephalus shunt.

Furthermore, the patent document US 2012/0265028 teaches a sensor arrangement based on a circular housing with a bottom wall which is configured as a diaphragm which is affected by the pressure within the brain.

Although the arrangements according to the two above-mentioned patent documents are suitable for measuring the pressure during hydrocephalus treatment, there is a need for further improvements of such systems.

A particular problem which may occur in pressure sensing systems of the above-mentioned kind relates to zero drift, i.e. a tendency for a zero reading of a sensor to drift and vary while the input signal is zero. Such a condition may arise over time and as a consequence of the environmental conditions in which the sensor operates. As a result, the measurement data may not be sufficiently accurate.

Furthermore, the environment in which sensor arrangements of the above-mentioned type are mounted and used, i.e. within the human brain, is demanding. For example, there is a risk for leakage of body fluids into the sensor arrangement, which may lead to deteriorated measurements and a decreased lifetime of the sensor arrangement.

For the reasons stated above, there is a desire to obtain improved and optimized arrangements and methods for measuring and monitoring intracranial pressure, for example in connection with hydrocephalus.

### SUMMARY OF THE INVENTION

In accordance with the disclosure, there is provided an improved sensor arrangement and corresponding method in which the drawbacks of known devices within this technical field can be overcome.

For this reason, and in accordance with this disclosure, there is provided a sensor arrangement for detecting an intracranial pressure, said arrangement comprising: a housing defining an internal space which is configured for accommodating a pressure sensor device and an electronic control unit to which said pressure sensor device is connected; and fastening means cooperating with said housing for permanently mounting said arrangement in a human skull bone. Furthermore, the housing is arranged with an opening which is covered by a membrane being made of titanium or a similar thin and flexible material, and is configured for generating vibrations in accordance with an existing outer pressure, said vibrations being transferred to said pressure sensor device.

Certain advantages are achieved by means of the device according to the disclosure. In particular, it should be noted that by means of the above-mentioned features, the titanium membrane can be configured so as to vibrate freely without any friction and transfer its movement, i.e. normally in the form of deflections, to accurate measurement data as registered by the pressure sensor device. Furthermore, the above-mentioned arrangement is based on a use of compact electronic units with short electric connections and a low risk for zero drift, which also contributes to accurate measurements. Also, the sensor arrangement can be arranged as a hermetically sealed unit.

According to an embodiment, the housing and opening are formed with a generally circular cross-section, and said membrane has a circular shape which corresponds to said opening.

According to an embodiment, said membrane has a thickness which is less than in the magnitude of 100 μm.

According to an embodiment, said membrane has a thickness which is within the interval of 5-50 μm.

According to an embodiment, the sensor arrangement comprises an electric accumulator arranged within said internal space.

According to an embodiment, the sensor arrangement comprises a charging arrangement which is configured for inductive charging of said electric accumulator.

According to an embodiment, the sensor arrangement comprises a charging arrangement based on an arrangement for energy harvesting.

According to an embodiment, the electric accumulator is based on a capacitor circuit.

According to a further embodiment, the housing is manufactured by titanium.

According to an embodiment, the pressure sensor device is mounted on a circuit board which is configured so as to divide the internal space into a first partial space between said circuit board and said membrane, and a second partial space between said circuit board and an end portion of said housing.

According to an embodiment the first partial space is filled with an incompressible medium, gas or vacuum.

According to an embodiment, the incompressible medium comprises at least one of liquid, oil or gel/silicon.

According to an embodiment, the pressure sensor device is mounted on said circuit board on a side which faces said membrane.

According to an embodiment, the electronic control unit is connected to a gyro.

According to an embodiment, the control unit is configured for calibrating the measurements of said pressure sensor device based on an existing atmospheric pressure.

According to an embodiment, said housing is formed with a section which is open in order to allow radio signal transmission.

According to an embodiment, said open section is covered with a sealing material such as ceramics, glass, epoxi material, silicon or a similar material.

The disclosure also relates to a method for detecting an intracranial pressure.

Further advantages and advantageous features of the embodiments contemplated herein are disclosed in the following description and in the dependent claims.

### BRIEF DESCRIPTION OF THE FIGURES

The disclosure will be described in greater detail below with reference to the figures shown in the appended drawings.

FIG. 1 shows a cross-sectional view of a part of a human brain, in which a sensor arrangement according to a first embodiment of the invention is mounted;

FIG. 2 shows a cross-sectional view of the sensor arrangement according to said first embodiment;

FIG. 3 shows a further cross-sectional view of the sensor arrangement;

FIG. 4 shows a perspective and cross-sectional view of the sensor arrangement which corresponds to the cross-sectional view of FIG. 2;

FIG. 5 show details of the sensor arrangement according to a modified embodiment; and

FIG. 6 shows further perspective views corresponding to FIG. 5.

### DETAILED DESCRIPTION OF PARTICULAR EMBODIMENTS

Different aspects of the present disclosure will be described more fully hereinafter with reference to the enclosed drawings and certain embodiments. The disclosure can be realized in many different forms and should not be construed as being limited to the embodiments below.

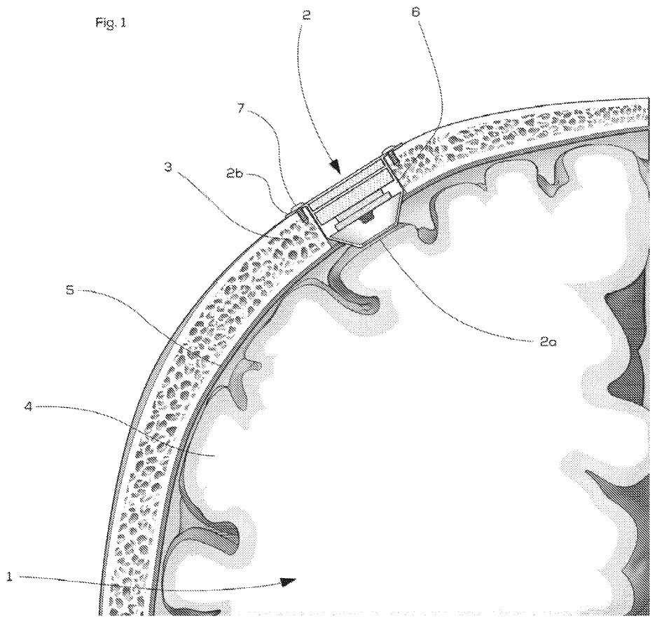

With initial reference to FIG. 1, there is shown a cross-sectional view of a section of a human skull 1, in which a sensor arrangement 2 according to an embodiment of the invention is mounted. Certain parts of the skull 1 are shown, such as the skull bone 3, the actual brain 4 with its cerebral cortex, and the dura mater 5, i.e. a hard membrane enclosing the brain 4.

As mentioned, the sensor arrangement 2 is configured for being mounted in the skull bone 3 in either a permanent or non-permanent manner. Suitably, it is mounted by first making a hole 6 in the skull bone 3 in a manner so that the inner dimensions of the hole 6 generally correspond to the outer dimensions of the sensor arrangement 2.

As will be described in greater detail below, the sensor arrangement 2 is configured with a lower end portion 2a which faces towards the brain 4 and also an upper end portion 2b which is formed as a flange, or rim, having a number of holes which are associated with a corresponding number of screws 7 or similar fastening elements. In this manner, a fastening arrangement for mounting the sensor arrangement 2 in its intended position as shown in FIG. 1 is defined. More precisely, the sensor arrangement 2 can be mounted (in a generally permanent manner, if desired) in the skull bone 3.

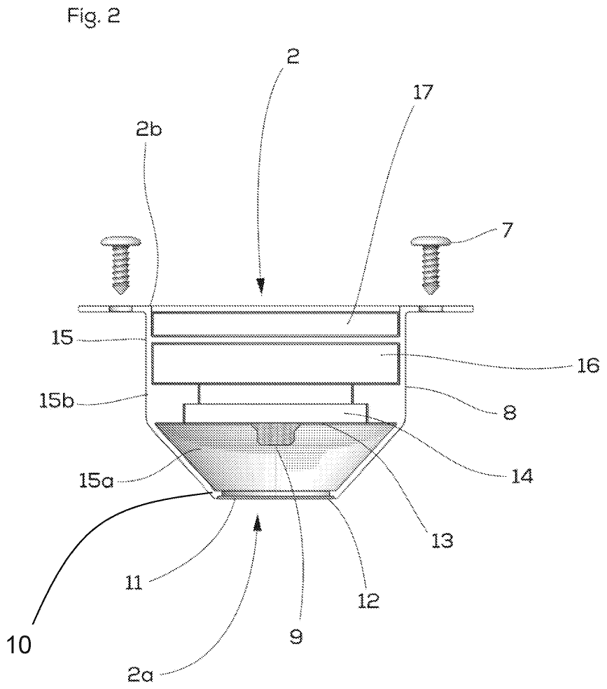

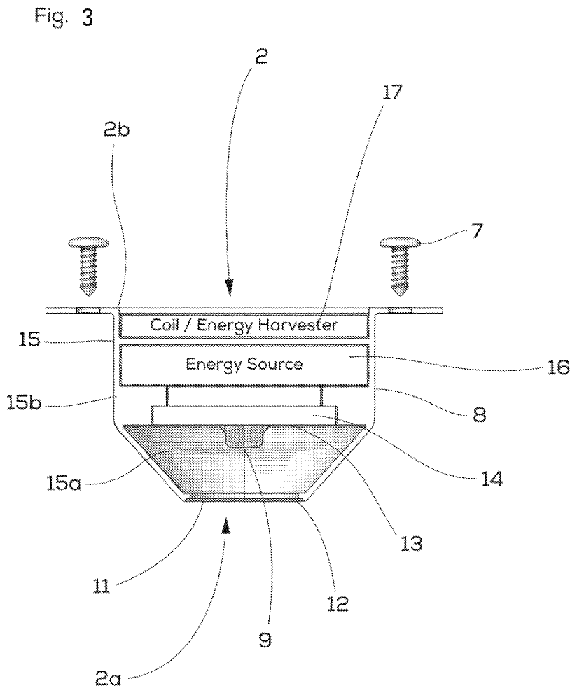

With reference to FIG. 2 and FIG. 3, there is shown in further detail how the sensor arrangement 2 is configured. According to an embodiment, the sensor arrangement 2 is based on a generally cylindrically shaped housing 8 which suitably is manufactured from titanium. The housing 8 is hermetically sealed, which means that no leakage from the outside, for example due to liquid in the brain 4, will be allowed into the housing 8. The housing 8 has a wall thickness which suitably is of the magnitude of 100-2000 μm. Also, according to an aspect, the diameter of the housing 8 is suitably in the magnitude of 5 -15 mm.

The housing 8 is connected to the upper end portion 2b of the sensor arrangement 2, i.e. the above-mentioned flange which is formed by the upper end portion 2b and which is used for fastening the housing 8, and thereby also the entire sensor arrangement 2, in its correct position as shown in FIG. 1.

The housing 8 is also connected to the lower end portion 2a of the sensor arrangement 2 and is configured for accommodating a pressure sensing device 9 within its interior. The pressure sensing device 9 is arranged so that it faces (during operation of the sensor arrangement 2) towards the brain 4 in order to measure the intracranial pressure therein, in a manner which will be described in more detail below.

The lower end portion 2a is formed with a generally circular end wall 10 which is formed with a preferably circular opening 11 in which a membrane 12 is mounted so as to cover the opening 11. According to an embodiment, the membrane 12 is generally circular and made of a thin, flexible material. Suitably, the membrane 12 is made by titanium and has a diameter which suitably is within the interval 4-8 mm, which generally corresponds to the diameter of the opening 11.

The purpose of the membrane 12 is to be influenced by the intracranial pressure and pulsations in the brain 4 so that movement of the membrane 12, for example in the form of regular or irregular deflections or vibrations, due to a varying pressure within the brain 4, will be transmitted towards the pressure sensing device 9 which is located within the interior of the housing 8. In this manner, pressure measurements can be registered in an accurate manner by means of the pressure sensing device 9.

According to an embodiment, the pressure sensing device 9 is mounted on an electric circuit, such as a printed circuit board 13, which is situated on the opposite side of the pressure sensing device 9, i.e. on the side of the pressure sensing device 9 which is not facing towards the brain 4. Also, the circuit board 13 is configured so that it supports an electronic control unit 14 to which the pressure sensing device 9 is electrically connected.

The electronic control unit 14 is programmed so as to control the measuring process relating to the intracranial pressure and to store and transmit measurement data supplied by the pressure sensing device 9. To this end, and according to an aspect, the electronic control unit 14 is based on a microprocessor unit which is associated with a memory unit (for storing measurement data), an input unit (for allowing input of measurement data) and an output unit (for transmitting measurement data to external components). The detailed implementation of the electronic control unit 14 is as such configured in accordance with generally known technology. For this reason, it is not described in greater detail here.

As shown in FIG. 2, the housing 8 defines, encloses and hermatically seals an internal cavity or space 15 which is divided into a lower internal space 15a and an upper internal space 15b. The lower internal space 15a and the upper internal space 15b are divided by means of the electric circuit board 13, which consequently has the function of a dividing wall. This also means that the pressure sensing device 9 is positioned within the lower internal space 15a. Suitably, the lower internal space 15a and the upper internal space 15b are sealed from each other, i.e. not connected.

According to an embodiment, the lower internal space 15a is filled with a non-compressible medium, such as suitably air, oil or silicon (in the form of a gel). When electronic components (such as the pressure sensing device 9) is enclosed within a hermetically sealed enclosure, there may occur pressure differences at different levels above the sea level. This may cause the thin membrane 12 to be deformed, which may lead to unwanted zero drift and possibly also damage to the membrane 12 due to such air pressure differences. By filling the lower internal space 15a with a non-compressible medium, a compensation for such air pressure differences will be provided.

Furthermore, as also indicated in FIG. 2, the printed circuit board 13 is connected to a power source 16, which suitably is in the form of an electric accumulator such as a chargeable battery, in order to supply electric power to the various components which are arranged on the printed circuit board 13, i.e. the electronic control unit 14 and its associated components. As shown in FIG. 2, the power source 16 is positioned in the upper internal space 15b, i.e. on the same side of the circuit board 13 as the electronic control unit 14.

The power source 16 is furthermore connected to a charging arrangement 17 which suitably is arranged within the upper internal space 15b. According to an embodiment, the charging arrangement 17 is of the energy harvesting type. Alternatively, the charging arrangement 17 can be based on a capacitor circuit with an inductive coil which is configured for inductive charging, i.e. wireless charging, via an external charging unit (not shown in the drawings).

FIG. 3 describes an aspect having an energy source 16 and a coil or energy harvester device 17.

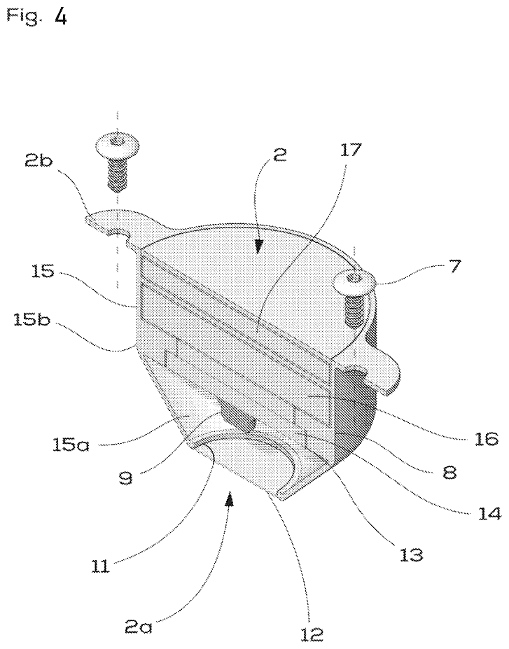

FIG. 4 is a perspective view showing the sensor arrangement in a perspective view and generally corresponding to FIG. 2 and FIG. 3.

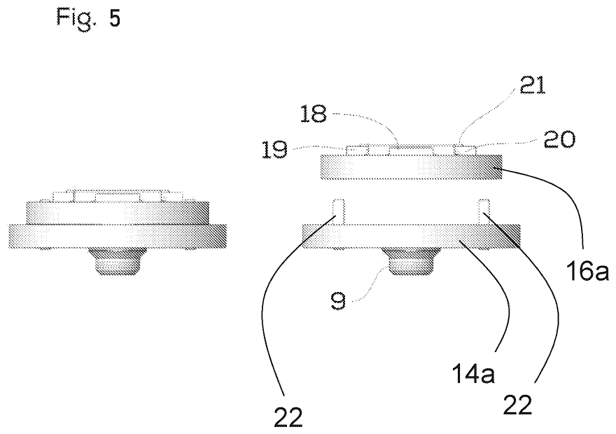

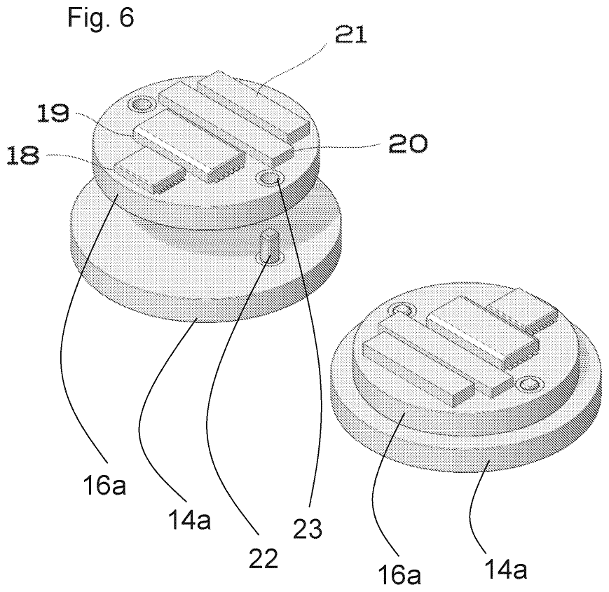

FIG. 5 and FIG. 6 show a further exemplary embodiment of how the sensor arrangement can be mounted. The embodiment is slightly modified as compared with FIGS. 1-5. More precisely, the control unit 14a (which generally corresponds to the control unit 14 as described above) can be provided with one or more pins 22. These pins 22 are arranged so as to be aligned with, and to cooperate with, corresponding holes 23 (see in particular FIG. 6) in the power source 16a. In this manner, the control unit 14a and the power source 16a can be mounted together with precision. With further reference to FIG. 5 and FIG. 6, the power source 16a also comprises certain electronic modules such as a central processing unit 18, a gyro sensor 19, an A/D converter 20 and an AC/DC converter 21. The central processing unit 18 can be arranged to operate together with the control unit 14a in a suitable way, i.e. one of these units may be a memory unit and the other may comprise other components such as a microprocessor, communication components and similar devices, or vice versa.

The electronic control unit 14a also comprises electronic connections to the power source 16a (not shown in the drawings). FIG. 5 also shows the pressure sensing device 9 (which is not shown in the view of FIG. 6). The control unit 14a and the power source 16a as shown in FIG. 5 and FIG. 6 are suitably generally circular and generally correspond to the interior dimensions of the housing 8. The gyro sensor 19 is previously known as such, and is used in order to provide information regarding the position of the user's head, in a three-dimensional space, when the sensor arrangement is implanted into the head.

According to an embodiment, which is not shown in the drawings, a portion of the upper end portion 2b is open, i.e. in order to allow radio transmission in and out of the housing 8. The opening is preferably sealed by means of a ceramic material, alternatively epoxy or silicon, in order to simplify the transmission of radio signals to and from the sensor arrangement while still sealing the housing 8. This is also implemented in order to allow inductive charging of the accumulator 16 as shown for example in FIG. 2.

In summary, and as shown in FIGS. 1-5, the disclosure relates to a sensor arrangement 1 for detecting an intracranial pressure. The arrangement 2 comprises a housing 8 defining an internal space which is configured for accommodating a pressure sensor device 9 and an electronic control unit 14 to which said pressure sensor device 9 is connected. The arrangement 2 also comprises fastening means (i.e. the screws 7 being associated with corresponding holes in the flange 2b) which cooperate with said housing 8 for mounting said arrangement 2, optionally permanently, in a human skull bone 3.

Furthermore, the housing 8 is arranged with an opening 11 which is covered by a membrane 12 being made of titanium and configured for generating vibrations in accordance with an existing outer pressure, said vibrations being transferred to said pressure sensor device 9. Furthermore, the pressure sensing device 9 is mounted on the underside of the circuit board 13, which leads to certain advantages. Initially, it should be mentioned that the pressure sensor device 9 provides very accurate and precise measurements without any substantial risk for zero drift. As shown in the embodiment in FIGS. 2-4, the housing 8 is suitably tapered along a section of the housing 8 which ends with the opening 11 and the membrane 12.

According to an embodiment, the pressure sensor device 9 is located fully within an internal space (see FIG. 2) which is formed by the housing 8 and the membrane 12 which covers the opening 11 and which, in this manner, forms a hermetically sealed housing arrangement which prevents passage of any liquid or gas. Suitably, the housing 8 is manufactured as one single piece, or unit. The pressure sensor device 9 does not form part of the actual housing or enclosure of the internal space but is arranged fully within the boundaries which are defined by means of the housing 8 and the membrane 12, i.e. within the interior of the housing 8. This also means that the membrane 12 and the pressure sensor device 9 are two separate components. Also, the membrane 12 is configured for transferring vibrations from outside the housing 8 (which causes movement of the membrane 12), via a compressible or non-compressible medium, and further to the pressure sensor device 9. Suitably, the medium is constituted by a gas.

Furthermore, according to an embodiment, the pressure sensor device 9 is suitably an active capacitive sensor device. The pressure sensor device 9 is controlled by the integrated electric circuit 14, to which it is connected, and is furthermore powered by the integrated energy source 16 (see FIG. 3).

As indicated in for example FIG. 2, the housing 8 and opening 11 are formed with a generally circular cross-section, whereas said membrane 12 has a circular shape which corresponds to said opening 11. Also, the membrane 12 has a thickness which is less than in the magnitude of 100 μm. According to an embodiment, the membrane 12 has a thickness which is within the interval of 5-50 μm.

The actual measurements and geometry of the sensor arrangements 2, i.e. as regards for example the dimensions of the housing 8, the fastening means 7, the pressure sensing device 9 and the membrane 12, may vary depending on the requirements for measurements and the medical conditions in question. According to an exemplary embodiment, the housing 8 may have a diameter of approximately 5 -15 mm, and the membrane 12 may have a diameter of approximately 4-8 mm. However, the invention is not limited to these specific dimensions.

Furthermore, as shown in FIG. 2, the sensor arrangement 2 comprises an electric accumulator 16 which is arranged within said internal space 15, and also a charging arrangement 17 which can be configured for example for inductive charging of said electric accumulator 16, or as an arrangement for energy harvesting.

Furthermore, the circuit board 13 is configured so as to divide the internal space 15 into a first partial space 15a between the circuit board 13 and the membrane 12, and a second partial space 15b between the circuit board 13 and an upper end portion 2b of the housing 8 of the sensor arrangement 2. According to an embodiment, the first partial space 15a is filled with a non-compressible medium such as for example at least one of air, oil or gel/silicon.

Also, the electronic control unit 14 is configured for calibrating the measurements of the pressure sensor device 9 based on an existing atmospheric pressure, which contributes to accurate measurement data.

According to a further aspect, the present disclosure also relates to method for detecting an intracranial pressure by means of a sensor arrangement 2. The method comprises a number of steps. Firstly, an internal space 15 is provided in a housing 8 forming part of the sensor arrangement 2. Furthermore, the internal space 15 accommodates a pressure sensor device 9 and an electronic control unit 14 to which the pressure sensor device 9 is connected.

Furthermore, the method comprises a step of mounting-optionally in a permanent manner-the arrangement 2 in a human skull bone 3 by means of fastening means 7; 2b which cooperate with the housing 8. Furthermore, the method also comprises the following steps:
an opening 11 in the housing 8 is provided;
the opening 11 is covered with a membrane 12 made of titanium or a similar thin and flexible material;
movement or deflections of the membrane 12 is allowed to be generated in accordance with an existing outer pressure; and
the movement of said membrane are transferred to the pressure sensor device 9 for measuring said outer pressure.

The invention is not limited to the embodiments described above but can be varied within the scope of the appended claims.

1. A sensor arrangement for detecting an intracranial pressure, said arrangement comprising:
a housing defining an internal space which is configured for accommodating a pressure sensor device and an electronic control unit to which said pressure sensor device is connected; and
fastening means cooperating with said housing for mounting, optionally in a permanent manner, said arrangement in a human skull bone;
wherein:
the housing is arranged with an opening which is covered by a membrane being made of titanium or a similar thin and flexible material, and is configured for generating vibrations in accordance with an existing outer pressure, said vibrations being transferred to said pressure sensor device.

2. A sensor arrangement according to claim 1, wherein said housing and opening are formed with a generally circular cross-section, and that said membrane has a circular shape which corresponds to said opening.

3. A sensor arrangement according to claim 1, wherein said membrane has a thickness which is less than in the magnitude of 100 μm.

4. A sensor arrangement according to claim 3, wherein said membrane has a thickness which is within the interval of 5-50 μm.

5. A sensor arrangement according to claim 1, further comprising an electric accumulator arranged within said internal space.

6. A sensor arrangement according to claim 5, further comprising a charging arrangement which is configured for inductive charging of said electric accumulator.

7. A sensor arrangement according to claim 1, further comprising a charging arrangement based on an arrangement for energy harvesting.

8. A sensor arrangement according to claim 5, wherein said electric accumulator is based on a capacitor.

9. A sensor arrangement according to claim 1, wherein said housing is manufactured by titanium.

10. A sensor arrangement according to claim 1, wherein said pressure sensor device is mounted on a circuit board which is configured so as to divide the internal space into a first partial space between said circuit board and said membrane and a second partial space between said circuit board and an end portion of said housing.

11. A sensor arrangement according to claim 10, wherein said first partial space is filled with an incompressible medium, gas or vacuum.

12. A sensor arrangement according to claim 11, wherein said incompressible medium comprises at least one of liquid, oil or gel/silicon.

13. A sensor arrangement according to claim 10, wherein said pressure sensor device is mounted on said circuit board on a side which faces said membrane

14. A sensor arrangement according to claim 1, wherein said electronic control unit is connected to a gyro.

15. A sensor arrangement according to claim 1, wherein said control unit is configured for calibrating the measurements of said pressure sensor device based on an existing atmospheric pressure.

16. A sensor arrangement according to claim 1, wherein said housing is formed with a section which is open in order to allow radio signal transmission.

17. A sensor arrangement according to claim 16, wherein said open section is covered with a sealing material such as ceramics, glass, epoxi material, silicon or a similar material.

18. A sensor arrangement according to claim 1, wherein the pressure sensor device is located fully within the internal space which is formed by said housing and the membrane covering the opening.

19. A sensor arrangement according to claim 1, wherein the internal space is formed by the housing and the membrane which covers the opening so as to form a hermetically sealed housing arrangement.

20. A method for detecting an intracranial pressure by means of a sensor arrangement, said method comprising:
providing an internal space by means of a housing forming part of said sensor arrangement;
accommodating, within said internal space a pressure sensor device and an electronic control unit to which said pressure sensor device is connected; and
mounting, optionally permanently, said arrangement in a human skull bone means of fastening means cooperating with said housing wherein:
providing an opening in said housing;
covering said opening with a membrane being made of titanium or a similar thin and flexible material;
allowing movement to be generated in said membrane in accordance with an existing outer pressure; and
transferring said movement to said pressure sensor device for measuring said outer pressure.
